(19) United States
(12) Patent Application Publication
(10) Pub. No.: US 2014/0073025 A1
(43) Pub. Date: Mar. 13, 2014

(54) METHOD AND APPARATUS FOR RECYCLING AND REUSING BEVERAGES AND OTHER LIQUIDS AND CONTAINER MATERIALS

(71) Applicant: TMD TECHNOLOGIES GROUP, LLC, Lafayette, LA (US)

(72) Inventor: TOMMY MACK DAVIS, SPARTANBURG, SC (US)

(73) Assignee: TMD TECHNOLOGIES GROUP, LLC, Lafayette, LA (US)

(21) Appl. No.: 14/025,355

(22) Filed: Sep. 12, 2013

Related U.S. Application Data
(60) Provisional application No. 61/700,049, filed on Sep. 12, 2012.

(51) Int. Cl. C12P 7/28; C12P 7/08; C12P 7/16

(52) U.S. Cl. CPC C12P 7/28; C12P 7/16; C12P 7/08
USPC 435/150; 435/160; 435/163

(57) ABSTRACT

A system for destruction and recycling of containers (such as bottles, cans and the like), as well as associated liquids, food products or other residue, greatly reduces waste discharge and/or disposal requirements. Packaging and other solids are shredded, ground or otherwise processed, while associated liquids are collected. Evaporation removes water for reuse, thus concentrating sugars and other food materials for utilization as agricultural amendments or stock for bio-product production. Bio-fuels/solvents are generated, typically using an Immobilized Microbe Bioreactor system modified for fuels/solvents production.

### CROSS REFERENCES TO RELATED APPLICATION

Priority of U.S. Provisional patent application Ser. No. 61/700,049, filed Sep. 12, 2012, incorporated herein by reference, is hereby claimed.

### STATEMENTS AS TO THE RIGHTS TO THE INVENTION MADE UNDER FEDERALLY SPONSORED RESEARCH AND DEVELOPMENT

None

### BACKGROUND OF THE INVENTION

1. Field of the Invention

The present invention pertains to a method and apparatus for the destruction and recycling of bottles, containers and related materials. More particularly, the present invention pertains to a method and apparatus for destruction and recycling of solid containers as well as associated liquids and/or residue, while significantly reducing or eliminating discharge of waste materials.

2. Brief Description of the Prior Art

Recycling of beverage bottles, containers and related materials currently involves many challenges. For example, solid items such as bottles, containers and/or other packaging are frequently made from many different materials which must be segregated or separated prior to recycling or reuse. Further, remaining liquids or other residue—which frequently have low pH—must be disposed of in an environmentally acceptable manner. Conventional methods of destroying and recycling such beverage bottles, containers and/or related materials generally result in creation of waste products requiring disposal.

Under existing laws and regulations, low pH materials such as soft drinks, juices and other similar goods, as well as some associated solid materials, have historically been treated as hazardous waste by waste generators. As a result, such materials typically must be disposed of in compliance with applicable laws and regulations which can include, without limitation, federal laws [such as Resource Conservation and Recovery Act (“RCRA”)] and state laws. Compliance with such laws and regulations can often greatly increase the expense and complexity of the disposal process.

However, if it is determined that such low pH materials do not constitute hazardous waste, but rather products having value, disposal of such low pH materials can often be exempt from many of said state and federal legal and regulatory requirements. Moreover, solid materials do not constitute solid wastes if they are recycled by being: (1) used or reused, not reclaimed, in an industrial process to make a product; (2) used or reused as an effective substitute for a commercial product; or (3) returned to the original process without reclamation or land disposal. Exemption from such state and federal legal and regulatory requirements can frequently drastically reduce the complexity and cost associated with a recycling process.

In order to qualify for such exemption, both a waste generator and recycler must generally demonstrate that such recycling is legitimate and complies with applicable documentation requirements. Both the waste generator and recycler must generally maintain adequate records to document their qualification for exemption, which may include but is not limited to, contracts, material safety data sheets, storage records, shipping papers, laboratory reports, waste profiles and/or records documenting use the product(s).

As noted above, conventional methods of destroying and recycling beverage bottles, containers and/or associated liquids and residue generally result in creation of waste products requiring disposal. Further, such conventional methods typically do not qualify for exemption from state and federal legal and regulatory requirements. As a result, such conventional methods are typically expensive, complex and result in creation of significant residual waste products requiring disposal or further handling.

Thus, there is a need for a method and apparatus for destroying and recycling solid materials (such as, for example, beverage bottles and/or other containers) as well as associated liquids and residue including, without limitation, out of date, off-spec, surplus, or returned drinks, juices, and/or related beverage and food products having a pH<1.0 and water soluble flammable components. The method and apparatus should beneficially account for a wide range of solid materials and associated liquids, and should result in minimal, if any, waste products. Remaining liquids or other residue—which frequently have low pH—should be converted, reused or disposed of in an environmentally acceptable manner.

### SUMMARY OF THE INVENTION

In accordance with the present invention, out of date, off-spec, surplus, or returned drinks, juices, and/or related beverage and food products (including, without limitation, those having a pH<1.0 and water soluble flammable components) are recycled for reuse with minimal, if any, resulting waste. Materials may be delivered in packaging (typically bottles, cans or cartons) or as bulk liquid. Packaged products can be processed through shredding and liquids separation facilities, while bulk delivery products can be sent directly to said liquids separation facility.

In accordance with the present invention, raw materials are unloaded and staged for introduction into a shredder. The received materials can be broken down to remove packing materials. Bottles can be placed loose into a cargo container and staged for shredding. Packing materials are beneficially collected and sent to plastic and cardboard recycle facilities for reuse, or for disposal. Wooden pallets are collected for reuse.

Raw products (including bottles described above) can be fed into a shredder that releases liquids which, in turn, are captured and stored in at least one catchment basin or tank. Shredded containers are sent to either bailer(s) (typically, for aluminum, polypropylene (“PP”), and high-density polyethylene (“HDPE”)), or to grinder(s) (typically, for polyethylene terephthalate (“PET”)). A water spray can be beneficially directed into such shredder to adjust the pH of any included liquid(s) and dissolve any water soluble components into the liquid, raising the flash point of the liquid (ideally above 40 degrees C.). After the raw product is shredded and liquids are separated, a certificate of destruction can be issued to a bottling/beverage company or other generator submitting the material in order to certify that the product(s) have been destroyed.

Bailed container products can be used in the production of new containers. Additionally, PET can be ground, washed and can be reused in the production of new PET containers. Wash water can be captured and added to liquids for water reclamation.

Captured liquids are stored in catchment tanks and transferred to a separation facility. In such separation facility of the present invention, vacuum evaporation removes water from such liquids for reuse, while concentrating sugars and other ingredients. Sugar and pulp contents of liquids (typically drinks and juices) are concentrated and the vacuum separated water is condensed and stored in clean water tanks. Clean water is suitable for industrial grade reuse and can be further filtered or otherwise processed to ultra-pure water for other use.

Concentrated sugar stored in concentrate tanks can be suitable for use as commercial feed stock product and as the primary feed stock in the production of bio fuels, and other high end bio-products. Specifically, sugar and pulp recovered by the present invention typically contains high fructose corn syrup, sucrose, and sometimes trace amounts of other sugar related materials suitable for fermentation. Sucrose is typically used in the production of ethanol only, while high fructose corn syrup can be used to produce a combination of ethanol, bio-butanol, and/or acetone.

The process of the present invention can use an immobilized microbe bioreactor (“IMBR”) as part of the production (typically fermentation) process. Such IMBR utilizes beneficial microbial population(s) immobilized on at least one bio carrier media as part of the production process. Such IMBR fermentation process can comprise elements of the methods and apparatuses disclosed in U.S. Pat. No. 8,227,219, as well as United States Patent Publication No. 2012/0301938, or some combination thereof, both of which are incorporated by reference herein for all purposes. After fermentation or other processing, bio fuel can be separated by distillation and treated (such as, for example) through a molecular sieve to produce bio fuels with high purity.

Bottoms from distillation column(s), should there be any, can be processed through a centrifuge. Reject material (typically solids containing pulp and/or bio mass) can be sent to either an aerobic digester to reduce odors or to an anaerobic digester to minimize the production of nuisance odors and to create methane, primarily for use in power generation. Gases produced in anaerobic digestion can be captured for use, such as in a third stage biofuels or other product generation facility in-line with other biofuels reactors.

Clarifier liquid can be returned to the liquids separation phase to concentrate sugars and produce industrial grade clean water. In a preferred embodiment, the method and apparatus of the present invention does not leave any significant waste materials that require disposal; all materials produce saleable products that can be commercialized or reused.

The method and apparatus of the present invention provide various benefits which can include, without limitation, the following:

1. Provides complete destruction and recycling of solids (including, without limitation, bottles and other containers) and related beverages and/or other liquids for reclamation;

2. Provides certification of destruction of submitted solids and liquids for generators;

3. Greatly reduces or eliminates landfill solids disposal requirements;

4. Greatly reduces or eliminates wastewater loading to existing waste water treatment facilities;

5. Can provide recycled aluminum and recycled plastics for reuse including but not limited to HDPE (high density polyethylene), PP (poly propylene), PET (polyethylene terephthalate));

6. Utilizes distillation including, but not limited to, vacuum distillation to separate and recapture water for industrial or other reuse;

7. Can purify water to higher industrial grades using a variety of filtration methods;

8. Utilizes IMBR technology to ferment and metabolize concentrated sugars and/or other materials;

9. Can produce ethanol, biobutanol, and/or acetone, as well as agricultural animal feed stock amendments, from mixed sugars and other waste materials;

10. Can utilize distillation to separate and purify biofuels and solvents from fermentation beer;

11. Can produce ultrapure bio-fuels/solvent products using methods including, without limitation, filtration;

12. Can utilize centrifugation to separate solids including, without limitation, residual microbial components from liquid remaining after distillation; and

13. Reuses water after centrifugation as a part of water recovery system.

### BRIEF DESCRIPTION OF DRAWINGS/FIGURES

The foregoing summary, as well as any detailed description of the preferred embodiments, is better understood when read in conjunction with the drawings and figures contained herein. For the purpose of illustrating the invention, the drawings and figures show certain preferred embodiments. It is understood, however, that the invention is not limited to the specific methods and devices disclosed in such drawings or figures.

FIG. 1 depicts a process flow schematic illustrating the method of the present invention.

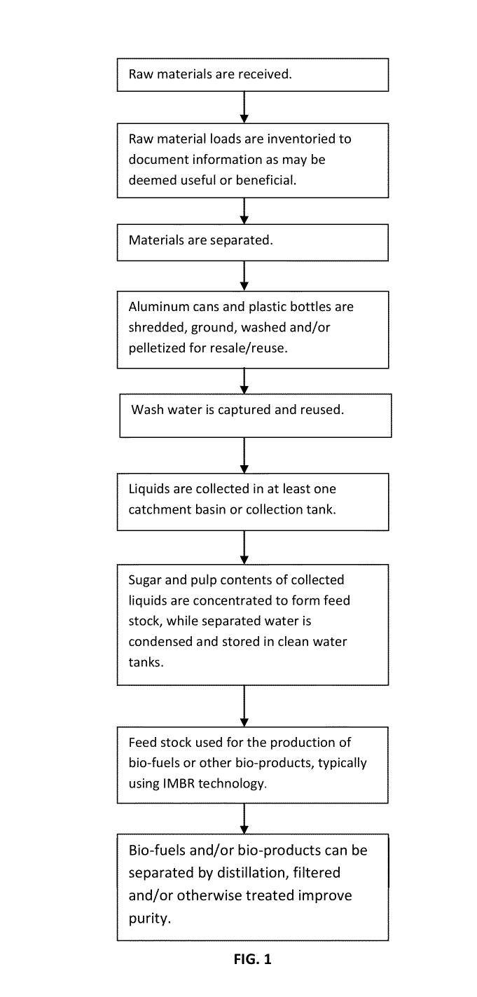

### DETAILED DESCRIPTION OF A PREFERRED EMBODIMENT

In a preferred embodiment, the method and apparatus of the present invention is used in connection with the recycling/reclamation of raw products comprising, without limitation, bottled/canned liquids, soft drinks and juices, as well as a wide range of related beverage and food products. Such raw product may be received in packaging, cases or as bulk liquid. As set forth more fully herein, packaged (typically, bottled/canned products) are generally processed through shredding and liquids separation facilities, while bulk liquids can be sent directly to said liquids separation facility.

In a preferred embodiment, raw products are received. Loads can be inventoried to document relevant information; such information can include, without limitation, receipt date, receipt time, transporter, generator (typically, a bottling/beverage company) sending the materials, packaging material, liquid content(s), amount of raw products, contact information and such other data or information as may be deemed useful or beneficial.

Shipping pallets, cardboard containers and other packaging materials can be manually opened and materials can be separated. Cans and bottles can be segregated and/or placed in holding bins for separate handling, while plastic sheeting and cardboard can collected and sent to plastic and cardboard recycling facilities for reuse. Wooden pallets or other shipping packaging can also be collected for reuse.

Aluminum cans and PP bottles can be shredded and bailed, while HDPE bottles can be shredded, ground, washed, pelletized. Recovered materials can be sold or otherwise provided to aluminum purchasers, plastics manufacturers or others for reuse. Liquids from said materials can be captured in at least one catchment basin or tank. Wash water is captured and reused for washing until it is no longer usable for such purpose; such water can be discharged via sewer after appropriate neutralization and solids separation and upon confirmation of meeting sewer standards.

Similarly, PET bottles can be shredded and/or ground. Shakers and cyclone technology for separation of fine dirt and light weight contaminants can be used as part of a separation process. Any remaining mixture of PET, labels, and caps can be sent to a hot water caustic bath to loosen and remove glue and dirt and to take advantage of density differentiation in order to separate different plastics. Glue and less dense plastics (such as, for example, from labels and caps) can float to the surface where they can be removed either manually or via a weir system; such materials can be recycled for future use. PET is allowed to sink for aggregation and collection. Wash water is re-circulated until no longer usable after which it is discarded via sewer after appropriate neutralization and solids separation and upon confirmation of meeting sewer standards.

Recovered PET material can be passed through a multi-stage agitation wash tank system to loosen remaining contaminants. A first bath can be used to loosen and remove contaminants. Thereafter a dryer, such as a vertical spin dryer, is used to remove wash water and loosened contaminants. Said PET materials can then be sent to a second agitation wash tank, through a hydrocyclone separator, and/or through a second spin dryer in order to remove water and remaining contaminants. Cleaned PET flakes can be further dried via additional spin dryers or other conventional methods. Dry materials can be visually and chemically analyzed prior to sale or other transfer to PET purchasers or other third parties.

After raw product is shredded and the liquid separated, a certificate of destruction can be issued to the waste generator (such as, for example, a bottling/beverage company) submitting any necessary material(s) certifying that the product has been destroyed.

All liquids from the various shredding systems are captured in a universal catchment basin for collection and storage prior to being sent to a vacuum separation unit. In said liquid separator, sugar and pulp contents of liquids, drinks, juices or other residue are concentrated. Any vacuum separated water is condensed and stored in clean water tanks.

Concentrated sugar stored in concentrate tanks can be suitable for use as commercial feed stock product and as the primary feed stock in the production of bio fuels, and other high end bio-products. Specifically, sugar and pulp recovered by the present invention typically contains high fructose corn syrup, sucrose, and sometimes trace amounts of other sugar related materials suitable for fermentation. Sucrose is typically used in the production of ethanol only, while high fructose corn syrup can be used to produce a combination of ethanol, bio-butanol, and/or acetone. Clean water is suitable for industrial grade reuse and can be further processed to ultra-pure water for industrial use via filtration or other conventional treatment methods.

The present invention can use at least one immobilized microbe bioreactor (“IMBR”) in order to produce bio fuels and/or other high end bio-products, typically using a fermentation process. Such IMBR utilizes beneficial microbial population(s) immobilized on at least one bio carrier media as part of the production process. Such IMBR process can comprise elements of the methods and apparatuses disclosed in U.S. Pat. No. 8,227,219, as well as United States Patent Publication No. 2012/0301938, or some combination thereof; both of which are incorporated by reference herein for all purposes. After fermentation or other production process, bio fuel can be separated by distillation and treated through a molecular sieve to produce bio fuels with high purity (in some cases up to 99.5%).

Bottoms from distillation column(s), should there be any, can be processed through a centrifuge. Reject material (typically solids containing pulp and/or bio mass) can be sent to either an aerobic digester to reduce odors or to an anaerobic digester to minimize the production of nuisance odors and to create methane, primarily for use in power generation. Gases produced in anaerobic digestion can be captured for use, such as in a third stage bio-fuels or other product generation facility in-line with other bio-fuels reactors. Clarifier liquids can be returned to the liquids separation phase to concentrate sugars and produce industrial grade clean water.

The method and apparatus of the present invention provide a means for destroying and recycling solid materials (such as, for example, beverage bottles and/or other containers) as well as associated liquids and residue including, without limitation, out of date, off-spec, surplus, or returned drinks, juices, and/or related beverage and food products having a pH<1.0 and water soluble flammable components. The method and apparatus of the present invention beneficially accounts for handling of a wide range of solid materials and associated liquids, and results in minimal, if any, residual waste products. Further, liquids or other residue materials processed with the present invention—which frequently have low pH—can be converted and/or reused in an environmentally acceptable manner. The method and apparatus of the present invention does not leave any significant waste materials that require disposal; all materials produce saleable products that can be resold, reused or otherwise commercialized.

The above-described invention has a number of particular features that should preferably be employed in combination, although each is useful separately without departure from the scope of the invention. While the preferred embodiment of the present invention is shown and described herein, it will be understood that the invention may be embodied otherwise than herein specifically illustrated or described, and that certain changes in form and arrangement of parts and the specific manner of practicing the invention may be made within the underlying idea or principles of the invention.

1. A method for recycling solid containers, beverages or other liquids comprising:
a) receiving a waste materials comprising at least one solid container and at least one liquid;
b) separating said at least one solid container from said at least one liquid;
c) concentrating sugars from said at least one liquids to create feed stock; and
d) generating bio-fuel from said feed stock.

2. The method of claim 1, wherein said step of separating said at least one solid container from said at least one liquid further comprises cutting said at least one solid container.

3. The method of claim 2, wherein said step of separating said at least one solid container from said at least one liquid further comprises collecting said at least one liquid in at least one catchment basin.

4. The method of claim 1, wherein said step of concentrating sugars from said at least one liquid to create feed stock comprises evaporating water from said at least one liquid.

5. The method of claim 1, wherein said step of generating bio-fuel from said feed stock comprises fermentation.

6. The method of claim 5, wherein said fermentation is performed using at least one immobilized microbe bioreactor.

7. The method of claim 5, wherein solids from the fermentation process are directed to an aerobic or anaerobic digester.

8. The method of claim 7, wherein gases produced in anaerobic digestion are used as fuel.

9. The method of claim 1, wherein said at least one solid container is recycled.

10. A method for recycling solid containers, beverages or other liquids resulting in substantially no residual waste products comprising:
a) receiving waste materials comprising at least one solid container and at least one liquid;
b) cutting said at least one solid container;
c) collecting said at least one liquid from said at least one container;
d) recycling said at least one solid container;
e) concentrating sugars from said at least one liquid to create feed stock; and
f) generating bio-fuel from said feed stock.

11. The method of claim 10, wherein said step of concentrating sugars from said at least one liquid to create feed stock comprises evaporating water from said at least one liquid.

12. The method of claim 10, wherein said step of generating bio-fuel from said feed stock comprises fermentation.

13. The method of claim 12, wherein said fermentation is performed using at least one immobilized microbe bioreactor.

14. The method of claim 12, wherein solids from said fermentation are directed to an aerobic or anaerobic digester.

15. The method of claim 13, wherein gases produced in anaerobic digestion are used as fuel.

16. The method of claim 10, wherein said at least one container comprises at least one plastic material.

17. The method of claim 16, wherein said at least one plastic material is shredded, ground and washed.

18. The method of claim 17, wherein said at least one plastic material is pelletized.
